(19) United States
(12) Patent Application Publication
(10) Pub. No.: US 2005/0059939 A1
(43) Pub. Date: Mar. 17, 2005

(54) Phacoemulsification needle

(76) Inventors: James T. Perkins, St. Charles, MO (US); Paul J. Ferreira, Stamford, CT (US)

Correspondence Address: Michael L. Smith;Bausch & Lomb Incorporated, One Bausch & Lomb Place, Rochester, NY, 14604-2701, US

(21) Appl. No.: 10/664,837

(22) Filed: Sep. 17, 2003

(51) Int. Cl. A61M 5/32

(52) U.S. Cl. 604/272

(57) ABSTRACT

A phacoemulsification cannula 10 includes a hub 18 for engagement with a surgical instrument 30. An elongated needle 12 has a proximal end 14 attached to the hub 18 and a distal end 16. The needle 12 has first and second inner diameters 22 and 24. The first inner diameter 22 is larger than the second inner diameter 24, and a transition 26 from the first inner diameter 22 to the second inner diameter 24 is closer to the proximal end 14 than to the distal end 16.

### BACKGROUND OF THE INVENTION

1. Field of the Invention

The present invention is directed to a phacoemulsification needle for use in ophthalmic surgery, particularly in cataract surgery. More specifically, the present invention is directed to a phaco needle having at least two (2) distinct inner diameters.

2. Description of Related Art

Phacoemulsification (phaco) needles are well-known in ophthalmic surgery for removing cataracts. Typically, a phaco needle has an inner diameter through which emulsified or chopped-up particles of a patient's cataractus lens are aspirated through the needle and a handpiece to a collection bag or reservoir. It has become common practice for the phaco needle to be vibrated with ultrasonic energy transmitted from a handpiece connected to the needle. This ultrasonic energy acts to break-up tissue, which tissue is then aspirated through the inner diameter of the phaco needle.

Different phaco needle constructions have been developed in the prior art to assist in breaking up a patient's cataract. For instance, Parisi in U.S. Pat. No. 4,816,018 discloses a phaco needle tip having a large inner diameter bore at a distal end of the phaco tip compared with a second smaller bore through out the remainder of the phaco needle. Such a larger inner diameter at the distal end of the phaco needle is provided to concentrate the ultrasonic energy within the larger bore of the needle to emulsify the bodily tissues more effectively then that accomplished by a constant diameter bored needle.

Likewise, U.S. Pat. No. 5,451,229 to Geuder, et al. teaches a stepped distal end of a phaco needle, wherein each successively smaller bore helps to further fragment a cataract tissue for aspiration through the needle. Another example of a multiple diameter phaco needle tip is Sutton, et al. in U.S. Pat. No. 6,533,750 that teaches the use of a conically shaped phaco tip for concentrating the ultrasonic energy for emulsifying a cataract tissue.

These prior art phaco needles with large diameter distal tips are effective at emulsifying tissue within the inner diameter of the tip but yet can have a repulsive effect that tends to push large lens fragments away from the phaco tip and does not allow the phaco needle to effectively bore deep into large fragments of the lens, particularly at lower vacuum settings. This characteristic of these needles makes the use of the common cataract surgery procedure called a chopping method to be more difficult to perform. Therefore, it would be desirable to have a needle that provides some of the advantages of the larger bore phaco tips but yet allows the phaco needle to more easily bore deep into large lens fragments.

### BRIEF DESCRIPTION OF DRAWINGS

FIG. 1 is a perspective view of a phaco needle in accordance with the present invention;

FIG. 2 is a cut away perspective view of a phaco needle in accordance with the present invention attached to a surgical handpiece;

FIG. 3 is a chart comparing a standard phaco needle to the present invention at varying vacuum levels;

FIG. 4 is a cut away elevation view of a phaco needle in accordance with the present invention;

FIG. 5 is a partial cut away elevation of an alternative embodiment in accordance with the present invention; and

FIG. 6 is a partial cut away elevation of yet another alternative embodiment in accordance with the present invention.

### DETAILED DESCRIPTION OF THE PREFERRED EMBODIMENT

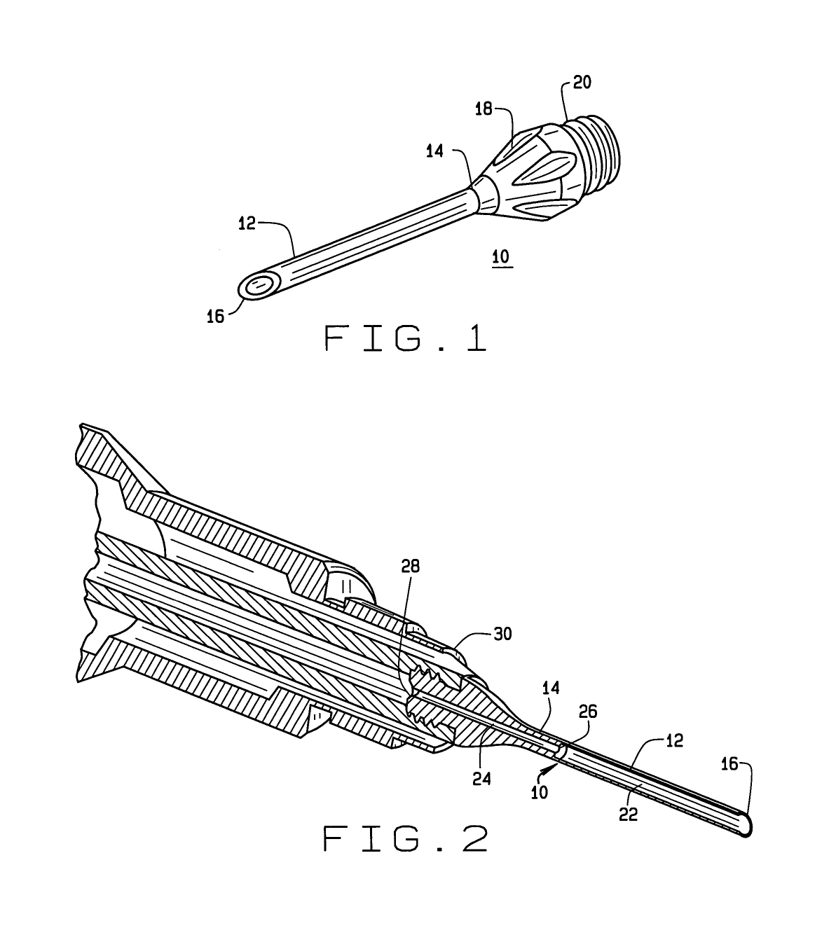

FIG. 1 shows a phaco needle or cannula 10 in accordance with the present invention. Phaco needle 10 includes a needle 12 having a proximal end 14 and a distal end 16. Proximal end 14 is attached to a hub 18, which includes threads 20 for engagement with a surgical instrument shown below. Attachment structures other than threads 20 may be used to attach cannula 10 to a surgical instrument.

FIG. 2 shows a cut away perspective view of the needle 10. Phaco cannula 10 includes the needle 12 having a first and a second diameter 22 and 24, respectively. As can be seen, the first inner diameter 22 is larger than the second inner diameter 24. In addition, a transition 26 from the first inner diameter 22 to the second inner diameter 24 is closer to the proximal end 14 than to the distal end 16. Another way to view cannula 10 is that it has an elongated needle 12 having a distal end 16 and a proximal end 28 structured for attachment to a surgical instrument 30, such as by threads 20 as shown in FIG. 2. In this view, it is still important that the transition 26 be closer to proximal end 28 than to distal end 16.

One of the main advantages of the present invention is to provide a reduced inner diameter that allows a surgeon to operate at a higher vacuum range, but yet with a lower flow rate. This combination of higher vacuum range and lower flow rate than is possible in some prior art needles, allows the surgeon to bore deep into a cataract and hold the cataract while a second instrument is used to segment the cataract (i.e. chopping). This deep bore is more easily achieved than in the prior art because of the transition being moved from near the distal end to near the proximal end of the cannula. This allows a surgeon to have superior vacuum holding power but yet have a low flow rate so that if problems were to occur, the surgeon has time to react before irreversible damage can occur.

Yet another advantage and benefit of the present invention is that the transition 26 from a large inner diameter to a small inner diameter provides a higher degree of ultrasonic displacement energy than if the larger inner diameter were toward the distal end 16. Placing the transition 26 as close to the surgical handpiece 30 as possible ensures that the aspirated tissue is completely emulsified and minimizes any possibility of clogging in the aspiration tubing (not shown). There is more ultrasonic energy at the transition from large to small diameter as the transition is moved closer to the source of the ultrasonic energy—the handpiece.

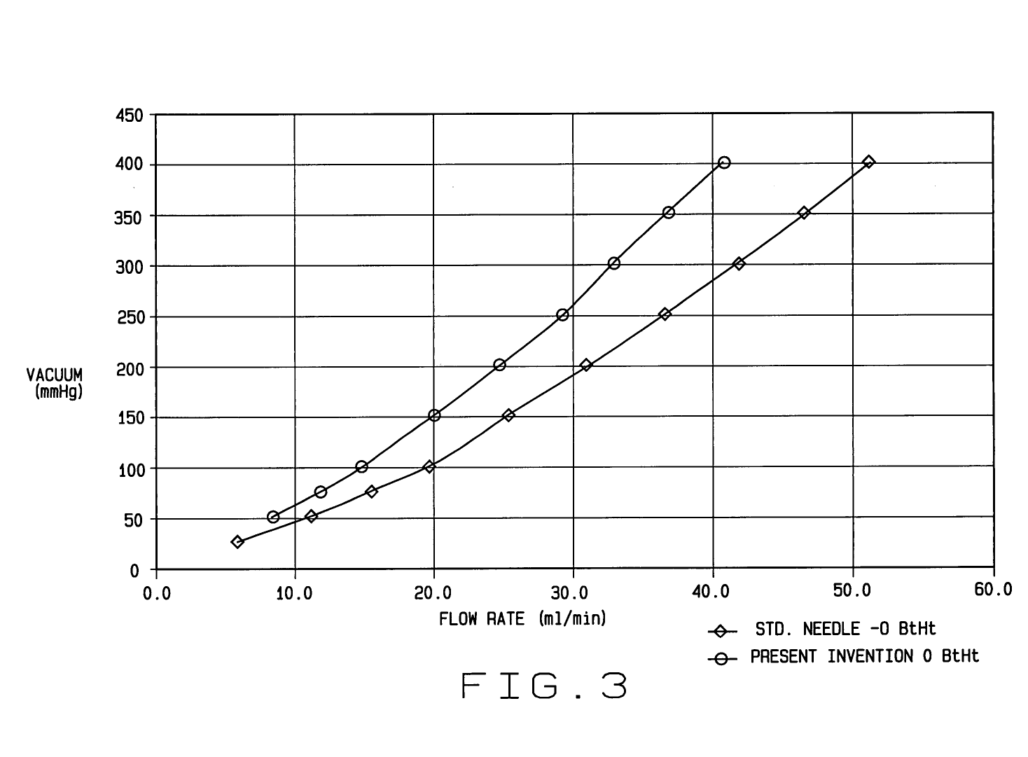

FIG. 3 shows a graph of vacuum in millimeters of mercury versus flow rate in milliliters per minute for both a standard phaco needle and the needle of the present invention at a zero (0) bottle height. As can be seen, the present invention provides for higher vacuum levels at any given flow rate as compared to a standard needle. This provides again, as stated above, the surgeon with greater holding power for the cataract at lower flow rates, which should ensure quicker and safer surgical procedures.

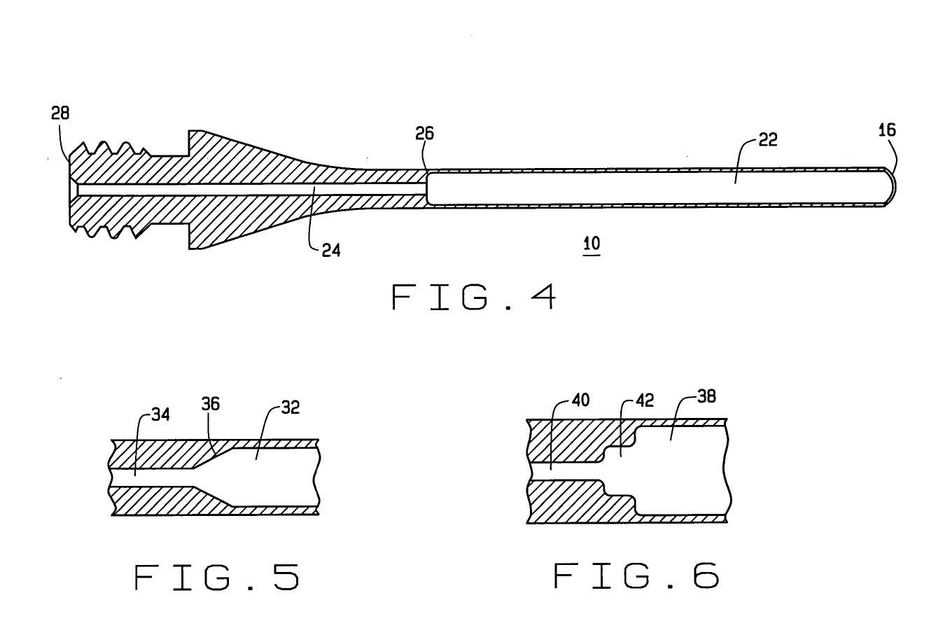

FIG. 4 shows phaco needle 10 including a first bore or inner diameter 22 extending from the distal end 16 toward the proximal end 28, a second bore or inner diameter 24 within the cannula 10 extends from the proximal end 28 to the first bore 22. As can be seen, the second bore 24 has a smaller diameter than the first bore. It is also preferable that second bore 24 is of sufficient length to provide a desired pressure drop during use across the length of the second bore 24. This pressure drop enables the user to use higher vacuum levels and still maintain relatively low flow rates. Also, it is preferred that the intersection or transition 26 of the first and second bores 22 and 24 is nearer the proximal end 28 than to the distal end 16.

As shown in FIG. 4, the transition 26 may simply be a radius. However, the transition between first bore 22 and second bore 24 may take on other configurations, such as those shown in FIGS. 5 and 6. FIG. 5 shows a partial needle depicting the transition between a first bore 32 and a second bore 34 having a conical transition 36. Likewise, FIG. 6 shows a transition from a first bore 38 to a second bore 40 having a stepped transition 42. Obviously multiple stepped transitions may also be used. Depending on the application and a particular surgeon's preference, the various transitions 26, 36, 42, or some other configuration may be used within a needle in accordance with the present invention.

1. A phacoemusification cannula comprising:
a hub for engagement with a surgical instrument;
an elongated needle having a proximal end attached to the hub and a distal end; and
the needle having a first and a second inner diameter wherein the first inner diameter is larger than the second inner diameter and wherein a transition from the first inner diameter to the second inner diameter is closer to the proximal end than to the distal end.

2. The needle of claim 1, wherein the transition includes a radius.

3. The needle of claim 1, wherein the transition includes a conical surface connecting the first and second inner diameters.

4. The needle of claim 1, wherein the transition includes at least one additional inner diameter stepped between the first and second inner diameters wherein the additional inner diameter is smaller than the first inner diameter and larger than the second inner diameter.

5. A phacoemulsification needle comprising:
an elongated needle having proximal and distal ends, wherein the proximal end is structured for attachment to a surgical instrument; and
the needle having a first and a second inner diameter wherein the first inner diameter is larger than the second inner diameter and wherein a transition from the first inner diameter to the second inner diameter is closer to the proximal end than to the distal end.

6. The needle of claim 5, wherein the transition includes a radius.

7. The needle of claim 5, wherein the transition includes a conical surface connecting the first and second inner diameters.

8. The needle of claim 5, wherein the transition includes at least one additional inner diameter stepped between the first and second inner diameters wherein the additional inner diameter is smaller than the first inner diameter and larger than the second inner diameter.

9. A phacoemulsification cannula comprising:
an elongated needle having proximal and distal ends, wherein the proximal end is structured for attachment to a surgical instrument,
a first bore within the cannula extending from the distal end toward the proximal end;
a second bore within the cannula extending from the proximal end to the first bore;
wherein the second bore has a smaller diameter than the first bore and is of sufficient length to provide a desired pressure drop during use across the length of the second bore and wherein an intersection of the first and second bores is nearer the proximal end than the distal end.

10. The needle of claim 9, wherein the transition includes a radius.

11. The needle of claim 9, wherein the transition includes a conical surface connecting the first and second inner diameters.

12. The needle of claim 9, wherein the transition includes at least one additional inner diameter stepped between the first and second inner diameters wherein the additional inner diameter is smaller than the first inner diameter and larger than the second inner diameter.
